(19) United States
(12) Patent Application Publication
(10) Pub. No.: US 2025/0120988 A1
(43) Pub. Date: Apr. 17, 2025

(54) SMALL MOLECULE COMPOUND FOR TREATING ISCHEMIC STROKE, ITS PREPARATION METHOD, AND APPLICATION

(71) Applicant: SHANGHAI SIXTH PEOPLE'S HOSPITAL, Shanghai (CN)

(72) Inventors: Shankai YIN, Shanghai (CN); Haibo SHI, Shanghai (CN); Ke LAI, Shanghai (CN); Hanwei LIU, Shanghai (CN); Zhenqi LIU, Shanghai (CN); Luyang WANG, Toronto (CA)

(21) Appl. No.: 18/398,167

(22) Filed: Dec. 28, 2023

(30) Foreign Application Priority Data
Oct. 16, 2023 (CN) 202311336286.6

(51) Int. Cl. A61K 31/675; A61P 9/10

(52) U.S. Cl. CPC A61K 31/675; A61P 9/10

(57) ABSTRACT

The present invention provides a small molecule compound for treating ischemic stroke, which is 4-(phenyl (phosphinoyl) methyl) piperazine-2-carboxylic acid. This small molecule compound can completely block the activation of ASICs by glutamate, and partially block the activation of NMDARs by glutamate, achieving the purpose of not affecting the physiological function of NMDARs, thereby reducing the side effects of mental symptoms and even mortality in stroke patients, and providing an effective strategy for treating ischemic stroke. In addition, the synthesis method of this small molecule compound is simple, which can reduce the cost of the pharmaceutical and has good application prospects.

### BACKGROUND OF THE INVENTION

### 1. Field of the Invention

The present invention relates to the field of biopharmaceutical technology, particularly to a small molecule compound for treating ischemic stroke, its preparation method and application.

### 2. Description of the Related Art

In the pathological process of ischemic stroke, it leads to local acidification of brain tissue and excessive release of the excitatory neurotransmitter glutamate. This will rapidly activate acid-sensing ion channels ((ASICs), which is mainly homotetramers or heterotetramers composed of ASIC1a subtype), and N-methyl-D-aspartate receptors (NMDARs). Both of them can be overactivated by glutamate, leading to rapid apoptosis and necrosis of neurons, further resulting in brain dysfunction and even death in patients. Therefore, the research on small molecule pharmaceutical S targeting ASICs and NMDARs for antagonizing stroke-induced brain injury has attracted increasing attention from researchers.

Currently, as inhibitors of ASIC1a and NMDARs, Selfotel (also known as CGS19755, with the chemical structure I shown as below) is a small molecule pharmaceutical that acts as a competitive antagonist, directly competing with glutamate to bind to the receptors. Selfotel has a molecular weight of 223.15 g/mol, which is a white powder, highly soluble in water, and can penetrate the blood-brain barrier to exert its effects in the brain. Early animal and clinical studies have shown that it has anticonvulsant, anxiolytic, analgesic, and neuroprotective effects, and can be used to treat brain damage caused by stroke.

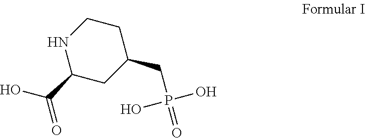 [SMILES: O=C(O)[C@@H]1C[C@H](CP(=O)(O)O)CCN1]

However, in clinical trials, Selfotel did not significantly reduce patient mortality compared to the control group and also led to various psychiatric symptoms in patients, such as mania, hallucinations, confusion, paranoia, and delirium. As a result, the Phase II clinical trial of Selfotel for the treatment of ischemic stroke was terminated. The analysis suggests that this is due to the important role of NMDARs in normal brain function, including cognitive and memory functions. Research has shown that NMDARs may even have a protective effect on neurons during stroke. Therefore, as a competitive antagonist of NMDARs, Selfotel's blocking effect is too strong, with an half-blocking dose (IC₅₀) less than 1 μM. This blocks both the physiological function and the neuroprotective effects of NMDARs, leading to the failure of the aforementioned clinical trial. Additionally, the binding site of Selfotel to ASIC1a, competing with glutamate, is unclear, making it difficult to develop more targeted and safer small molecule compounds.

Therefore, there is an urgent need to develop a novel small molecule compound that can completely block the activation of ASICs by glutamate while partially blocking the activation of NMDARs by glutamate.

### SUMMARY OF THE INVENTION

In response to the issues present in existing technologies, the invention provides a small molecule compound for treating ischemic stroke, its preparation method and application.

To achieve the above mentioned purpose, the technical solution is listed as follows.

In the first aspect of the invention, there is provided a small molecule compound for treating ischemic stroke, wherein the small molecule compound is 4-(phenyl (phosphinoyl) methyl) piperazine-2-carboxylic acid, and its structural formula is as follows:

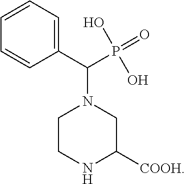 [SMILES: O=C(O)C1CN(C(c2ccccc2)P(=O)(O)O)CCN1]

In the second aspect of the invention, there is provided a method for preparing the small molecule compound as mentioned above, comprising:
step 1, synthesis of 1-benzyl-2-methyl-4-((dimethoxyphosphoryl)(phenyl)methyl)piperazine-1,2-dicarboxylate: dissolving 2-methylpiperazine-1,2-dicarboxylate benzyl ester in an organic solvent, benzaldehyde, and dimethyl phosphite, and reacting to obtain 1-benzyl-2-methyl-4-((dimethoxyphosphoryl)(phenyl)methyl)piperazine-1,2-dicarboxylate;
step 2, synthesis of 4-(phenyl(phosphinoyl)methyl)piperazine-2-carboxylic acid: dissolving 1-benzyl-2-methyl-4-((dimethoxyphosphoryl)(phenyl)methyl)piperazine-1,2-dicarboxylate in concentrated hydrochloric acid, and reacting to obtain 4-(phenyl(phosphinoyl)methyl)piperazine-2-carboxylic acid.

Furthermore, the organic solvent is acetonitrile.

Furthermore, in step 1, the molar ratio of 2-methylpiperazine-1,2-dicarboxylate benzyl ester, benzaldehyde, and dimethyl phosphite is 1:1˜1.5:1.25˜1.80; preferably 1:1.09:1.29.

Furthermore, the reaction condition for step 1 is at 70˜90° C. lasting for 36˜60 hours, preferably at 80° C. lasting for 48 hours.

Furthermore, in step 2, the molar ratio of 1-benzyl-2-methyl-4-((dimethoxyphosphoryl)(phenyl)methyl)piperazine-1,2-dicarboxylate to hydrochloric acid is 5˜10:1, preferably 7:1.

Furthermore, the reaction condition for step 2 is at 80˜100° C. lasting for 18˜36 hours, preferably at 90° C. lasting for 24 hours.

Furthermore, step 1 further comprises: when the reaction reaches its end, the solvent is removed by using a rotary evaporator under reduced pressure; the resulting residue is purified by column chromatography.

Furthermore, step 2 further comprises: when the reaction reaches its end, the reaction mixture is cooled to room temperature, concentrated to dryness by using a rotary evaporator under reduced pressure; the resulting residue is purified by reverse-phase chromatography.

In the third aspect of the invention, there is provided an application of the small molecule compound as mentioned above in the preparation of a pharmaceutical for treating ischemic stroke, wherein the pharmaceutical comprises the small molecule compound which functions as an effective active ingredient.

In the fourth aspect of the invention, there is provided a competitive antagonist of acid-sensing ion channel proteins (ASICs), comprising the small molecule compound as mentioned above.

In the fifth aspect of the invention, there is provided a pharmaceutical or pharmaceutical composition for treating ischemic stroke, comprising the small molecule compound as mentioned above which functions as an effective active ingredient.

The invention has the following advantages in comparison of the prior art:
the invention provides a small molecule compound for treating ischemic stroke which can not only block the activation of ASICs by glutamate, but also partially block the activation of NMDARs by glutamate, achieving the purpose of not affecting the physiological function of NMDARs, thereby reducing the side effects of mental symptoms and even mortality in stroke patients, and providing an effective strategy for treating ischemic stroke. In addition, the synthesis method of this small molecule compound is simple, which can reduce the cost of the pharmaceutical and has good application prospects.

### BRIEF DESCRIPTION OF THE DRAWINGS

FIG. 1 is a schematic diagram of the process for screening small molecule compounds in an embodiment of the present invention, where MW represents the relative molecular weight of the compounds; S log P represents the octanol/water partition coefficient, reflecting the lipophilicity of the compounds, with larger values indicating better lipid solubility of the compounds; TPSA represents the sum of the surfaces of polar atoms of the molecules, with smaller values indicating stronger ability of the compounds to pass through the blood-brain barrier.

FIG. 2 shows the dose-response curve of competitive blocking of ASIC1a current by LK-2. Figure a illustrates the LK-2 concentration-dependent blocking of signal of ASIC1a current (recorded from the same CHO cell) activated at pH 7 in patch clamp experiments, with a glutamate concentration of 500 μM and LK-2 concentrations of 1, 10, 50, and 100 μM in sequence. Figure b displays the dose-response curve fitted based on the current amplitudes recorded in Figure a, with IC₅₀ representing the half-maximal inhibitory concentration. Smaller values indicate stronger blocking effects of the compound.

FIG. 3 shows the dose-response curves of competitive blocking of NMDAR currents by LK-2 on different subtypes. Figure a illustrates LK-2 concentration-dependent blocking of signal of GluN1+GluN2A and GluN1+GluN2B subtypes of NMDAR currents (recorded from the same CHO cell) in patch clamp experiments, with 100 μM NDMA and 1 μM glycine used to activate NMDAR currents. LK-2 concentrations were 1, 10, 100, and 500 μM in sequence. Figure b illustrates the dose-response curves fitted based on the current amplitudes recorded in Figure a, with IC₅₀ representing the half-maximal inhibitory concentration. Smaller IC₅₀ values indicate stronger blocking effects of the compound.

FIG. 4 shows a schematic diagram of the process of neuronal cell viability assay.

FIG. 5 shows the results of cell viability-death staining and lactate dehydrogenase (LDH) release detection under in vitro simulated ischemic pathology (OGD) conditions. Figure a illustrates cortical neurons staining from wild-type (ASIC1a+/+) and gene-deficient (ASIC1a−/−) mice primary cultures in the calcein-PI staining experiment. Blue green represents viable cells, and red represents dead cell nuclei. 10 μM CGS19755 and 10 μM LK-2 were added respectively in the cell culture medium under pH 7.0 conditions with OGD to be treated for 1 hour. Figure b illustrates the statistical analysis of cell death percentages in the experimental groups shown in Figure a. Figure c illustrates, after being treated in the same experimental groups as Figure a, the analysis of each group of LDH of relative release levels. Figure d illustrates PI staining of ASIC1a-transfected CHO cells after 1 hour of treatment with pH 7.0 and 500 μM glutamate (Glu). Green represents green fluorescent protein (GFP), indicating ASIC1a-expressing CHO cells (GFP+), and red represents dead cell nuclei. The concentration of LK-2 was 10 μM. Figure e illustrates the cell death percentages in the experimental groups shown in Figure d. GFP-indicates ASIC1a-untransfected cells.

FIG. 6 shows the results of microscale thermophoresis detection. Figure a illustrates a representative binding curve of microscale thermophoresis, with the left vertical bar indicating cold fluorescence (−1˜0 seconds) and the right vertical bar indicating hot fluorescence (4˜5 seconds). Figure b illustrates the dose-response curve of LK-2 binding to ASIC1a, with K_{d} representing the dissociation constant. A smaller K_{d} value indicates a stronger binding of the small molecule to the protein.

FIG. 7 shows the cerebral infarction volume in mice with different drug treatments under in vivo simulated cerebral ischemia pathology (MCAO) condition. Figure a illustrates the TTC staining results of brain slices obtained from animals sacrificed 24 hours after different drug treatments following MCAO ischemia for 30 minutes. The dark areas represent normal brain tissue, while the white areas represent infarcted brain tissue. CGS19755 was administered at a concentration of 1 mg/kg (intraperitoneal injection), PcTX-1 was administered at a concentration of 100 ng/kg (ASIC1a blocker, intranasal drip), LK-2 was administered at concentrations of 10 mg/kg or 30 mg/kg (intraperitoneal injection), and Saline was used as the control group. Figure b shows the statistical analysis of the cerebral infarction volume in each group of model animals.

FIG. 8 shows the results of drug intervention and brain function testing under in vivo simulated cerebral ischemia pathology (MCAO). Figure a illustrates the experimental process, where drug intervention (using a concentration of 30 mg/kg of LK-2) was conducted 30 minutes after modeling, and the first brain function test (rotarod test) was performed 24 hours after modeling. The second brain function test was conducted 7 days later, with 5 training tests followed by 1 formal test in each rotarod test. Figure b illustrates the time taken for mice to fall off the rotarod during the 5 training tests, where longer time indicates better motor function. The sham surgery group exhibited the strongest motor learning ability, while both the LK-2 intervention group and the physiological saline control group showed a significant decline in motor learning ability 24 hours after modeling. The LK-2 intervention group showed a significant improvement in motor learning ability 7 days after modeling, while the physiological saline control group did not show significant improvement. Figure c illustrates the analysis results of motor ability in the formal test conducted 24 hours and 7 days after modeling for each group of mice.

### DETAILED DESCRIPTION

The present invention is further described in detail and with specific examples and accompanying figures below, in order to provide a better understanding of the invention. However, it should be understood that the following examples do not limit the scope of the invention.

In the implementation examples, unless otherwise specified, conventional methods are used. Unless otherwise specified, commercially available reagents or reagents prepared according to conventional methods are used.

### Example 1

The present example describes a novel small molecule compound that can completely block the activation of ASIC1a by glutamate and partially block the activation of NMDARs by glutamate. Using computer-aided drug design, the small molecule compound is screened based on the structure of Selfotel (CGS19755) using a scaffold replacement method. The specific design and screening steps are as follows:
1. Referring to FIG. 1, the MOE software was used to screen the ZINC20 database (fragment groups, lead-like compounds, and drug-like small molecules), and the screened 222 compounds were used to construct the three-dimensional structures of the compounds using the LigPrep module in Maestro software (OPLS3e force field, protonated at pH 7.0). The constructed compounds were subjected to high-throughput molecular virtual screening using the Glide module with high precision molecular docking, resulting in 6 potential compounds for synthesis, including LK-2 (4-(phenyl(phosphoryl)methyl)piperazine-2-carboxylic acid, C₁₂H₁₇N₂O₅P, with a relative molecular mass of 300.25 g/mol, as shown in the structure formula II). LK-2 was then subjected to electrophysiological and biological experiments to verify its suitability.

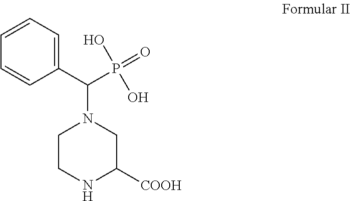 [SMILES: O=C(O)C1CN(C(c2ccccc2)P(=O)(O)O)CCN1]

2. Electrophysiological experiments refer to the use of patch clamp techniques to overexpress ASICs (ASIC1a) and NMDARs (GluNR1+NR2A or GluNR1+NR2B) in CHO cell lines (Chinese hamster ovary cells), with the above-mentioned proteins carrying GFP green fluorescent protein for identification under fluorescence microscopy. The synthesized small molecules are separately tested for their blocking effects on the currents generated by the two types of receptors, and the IC50 values are calculated.
2.1 The experimental steps of the membrane clamp technique are as follows.

Whole-cell patch clamp recordings were performed on neurons or CHO cells cultured in a cell-culture dish mounted on a fixed stage upright microscope. The recording electrode (resistance 4-6 MΩ) was made of 1.5 mm borosilicate glass. The EPC-10 patch clamp amplifier (HEKA) was used to record whole-cell currents. Data was sampled at a frequency of 10-20 kHz and the noise was filtered at 1-3 kHz using the Pulse 6.0 software (HEKA) on a computer. Unless otherwise stated, recordings were made at a holding potential of −60 mV. For NMDAR current recordings, the holding potential was −70 mV. A multi-channel perfusion system (SF-77B) and pressure control system (ALA-VM8) were used to achieve rapid exchange of extracellular solutions. The perfusion program was set and controlled using the PatchMaster software. To prevent dependent desensitization, ASIC currents were repeatedly activated one time by acidic solution at least every 1 minute. The composition and concentration of the physiological extracellular solution (ECS) were as follows: 140 mM/L NaCl, 5 mM/L KCl, 1 mM/L CaCl₂, 1 mM/L MgCl₂, 10 mM/L glucose, and 25 mM/L HEPES (pH buffer), which were adjusted to the desired pH with NaOH. The composition and concentration of the intracellular solution for ASIC1a current recordings were (all concentrations in mM/L): 120 potassium gluconate, 2 MgCl₂, 1 CaCl₂, 10 HEPES, 0.5 EGTA, 4 Mg-ATP, pH 7.3 (adjusted with KOH); for NMDAR current recordings, potassium gluconate in the intracellular solution was replaced with an equimolar concentration of CsCl (pH adjusted with CsOH). The osmolarity of all solutions was 290-320 mOsm, and the temperature of the patch clamp experiments was maintained at room temperature (approximately 25° C.).
2.2 The specific steps of cell transfection are as follows:

Chinese hamster ovary cells (CHO) and HEK293T cells (provided by the Chinese Academy of Sciences Stem Cell Bank) were cultured separately in F12 and DMEM media (Gibco) containing 10% FBS (Gibco). Penicillin/streptomycin (Invitrogen, final concentration 1%) was added to the culture media to prevent bacterial contamination. For NMDAR expression, 500 μM D-AP5 was added to the culture media to avoid glutamate toxicity caused by NMDAR activation. 0.25% trypsin-EDTA (Gibco) was used for cell passage. Cells were cultured in a homothermal CO₂ incubator at 37° C. At 50%˜70% cell density, a total of 3.0 μg cDNA was mixed with Lipofectamine 3000 transfection reagent (Invitrogen) and the mixture was added to a 35 mm culture dish. For NMDAR expression, plasmids for GluN1, GluNR2A or GluNR2B and PSD-95 were used in a ratio of 1:4:0.5. The cDNA for green fluorescent protein (GFP) was linked to the N-terminus of these proteins. All experiments were performed 24-48 hours after transfection, and all culture dishes were washed three times with ECF before the experiment, and GFP-positive CHO cells were observed under a fluorescence microscope for patch clamp recording. All overexpressed proteins were of human origin.

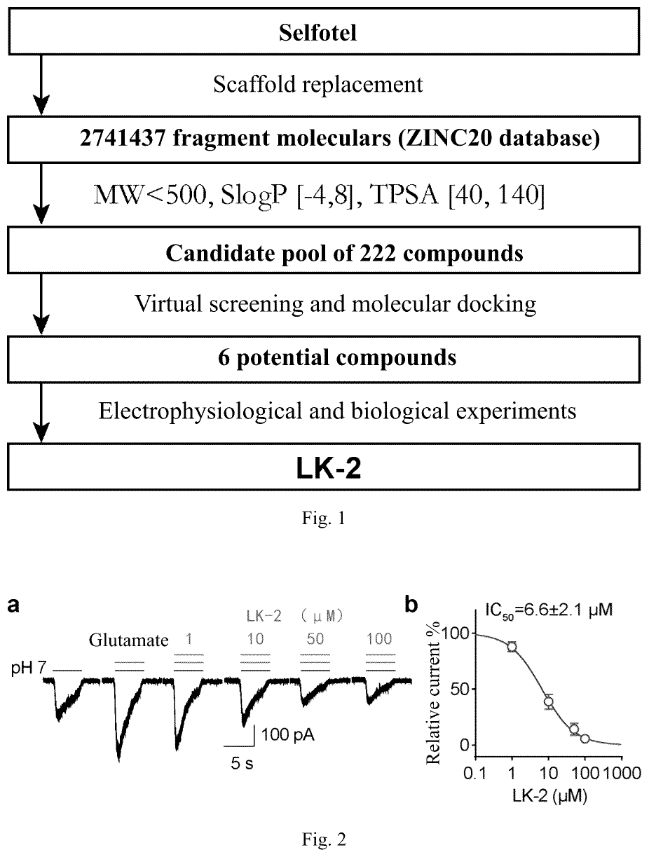

As shown in FIG. 2, the IC₅₀ of LK-2 competitive antagonism against the glutamate binding ASIC1a current is 6.6±2.1 μM (at pH 7.0).

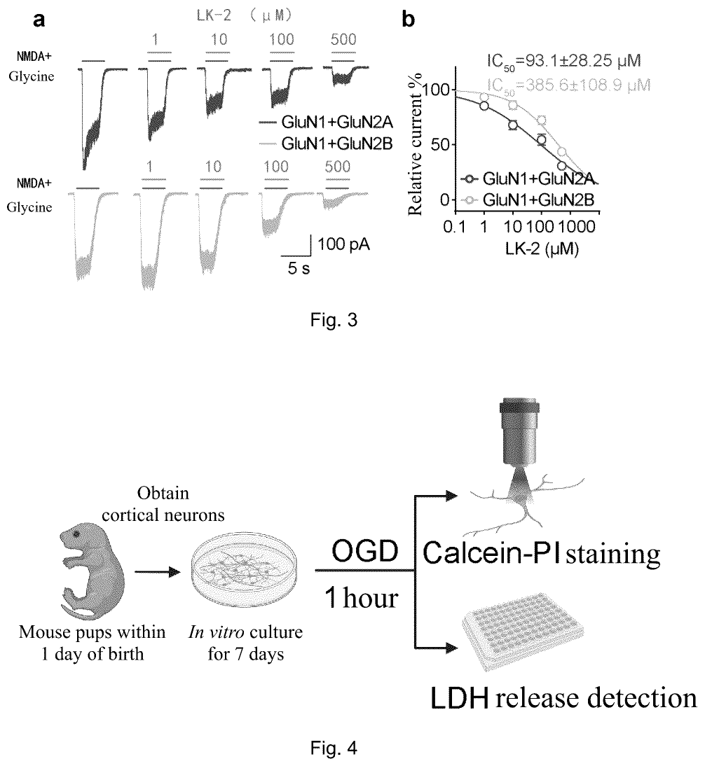

As shown in FIG. 3, the IC₅₀ of LK-2 competitive antagonism against glutamate binding GluN1+NR2A currents is 93.1±28.25 μM, and the IC₅₀ of LK-2 competitive antagonism against glutamate binding GluN1+NR2B currents is 385.6±108.9 μM. These results indicate that LK-2 can significantly inhibit the activation of ASIC1a by glutamate, while slightly inhibiting the function of NMDAR.
3. Biological experiments include cell viability assays, protein-small molecule binding analysis, pharmacokinetic studies of LK-2 small molecules, and animal models of cerebral ischemic stroke.
3.1 Cell viability assay

The process of viability assay of neurons is illustrated in FIG. 4, and the specific steps are as follows.
(1) Primary neuron culture and oxygen-glucose deprivation model ((OGD) used to simulate neuronal damage during brain ischemia) steps: preparation and culture of primary cortical neurons, dissecting the cortex of wild-type or ASIC1a gene knockout mice at 24 hours after birth in DMEM high glucose solution (Gibco), and dissociating with 0.05% trypsin (Gibco) for 15 minutes. Seeding the cells on coverslips or culture dishes coated with matrix gel (Corning) (approximately 2×10⁵ cells/35 mm culture dish for electrophysiology and cell death experiments). Culturing the neurons in Neurobasal-A culture medium (Gibco) supplemented with 2% B27 (Gibco) and 1% glutamine supplement (Gibco), and placing the culture dish in a 37° C., 5% CO₂ incubator. After 7 days of neuron culture, OGD is performed. The specific steps are as follows: washing the cultured neurons three times with glucose-free ECF (glucose replaced with equimolar concentration of sucrose), re-incubating in glucose-free ECF at pH 7.0, and dividing into control group and drug intervention group. In the drug intervention group, 10 μM CGS19755 or 10 μM LK-2 was added, and the culture dishes thereof are placed in a 35° C. hypoxic incubator (95% N₂+5% CO₂, controlled by BioSpherix software) together with the control group culture dishes. After 1 hour, OGD is terminated by replacing the glucose-free ECF with normal ECF.
(2) Cell viability-death (calcein-PI) staining and lactate dehydrogenase (LDH) release detection (for detecting cell viability status) procedure: for cultured neurons, after 1 hour of oxygen-glucose deprivation (OGD), perform calcein-PI cell death detection, and expose neurons to 1 μM calcein-am and 2 μM PI for incubating for 15 minutes (ECF pre-saturated with 95% O₂−5% CO₂).

Taking into consideration that in addition to ASIC1a, neurons also express multiple membrane proteins that can be activated and lead to cell death during OGD, in order to more accurately verify that cell death is caused by glutamate activation of ASIC1a and that LK-2 indeed competitively antagonizes the glutamate so as to reduce cell death, we conducted the following experiment: CHO cells transfected with ASIC1a-GFP were divided into a control group (500 μM glutamate) and a drug intervention group (500 μM glutamate+10 μM LK-2). After 1 hour of treatment in pH 7 ECF, PI detection was performed on both groups of cells, and CHO cells were exposed to 2 μM PI staining for 15 minutes only. Then, all cells were washed three times with ECF and fixed in 4% paraformaldehyde for 30 minutes, followed by obtaining images using a confocal microscope. Live and dead cell nuclei were determined by observing under a 40× objective using excitation wavelengths of 488 nm and 546 nm. The number of stained cell nuclei was counted using Image-J software.

The LDH assay kit (Biyuntian) was used to measure the release of LDH from cultured neurons after 1 hour of oxygen-glucose deprivation (OGD). After OGD incubation, the culture medium (60 μl) was transferred to a 96-well plate and mixed with 30 μl of the provided reaction solution from the kit, followed by a 30-minute incubation. The absorbance of the target was measured at 490 nm wavelength using a spectrophotometer (Thermo Scientific), while the background absorbance was measured at 620 nm wavelength. The maximum LDH release for each cell group was obtained by incubating with 1% Triton-100 for 30 minutes.

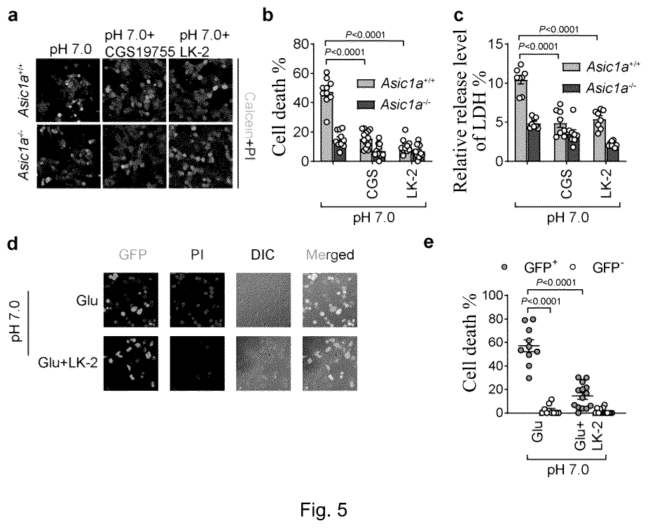

As shown in FIG. 5, for primary cultured neurons, 10 μM LK-2 reduced cell death rate by 87% and lactate dehydrogenase release by 50%, indicating that LK-2 significantly improves OGD-induced cell death. For CHO cells transfected with ASIC1a, 10 μM LK-2 reduced the death rate of transfected cells by 40%, suggesting that LK-2 provides cellular protection in an in vitro ischemia model.
3.2 The technique used for protein-small molecule binding analysis is Microscale Thermophoresis (MST) technology, which is employed to detect the binding of the target protein with a fluorescence (GFP) and small molecule compounds in cell lysates. The value of the binding constant K_{d} or the value of the median effective dose (EC₅₀) were analyzed. The specific steps are as follows: NT.115 (NanoTemper) is used to detect the binding of the compound with ASIC1a. HEK293T cells transfected with wild-type ASIC1a plasmid tagged with eGFP were collected after 48 hours and lysed using mammalian protein extraction reagent (Thermo). Protease/phosphatase inhibitors (1% concentration) were added to the lysate to prevent protein degradation. The expression level of ASIC1a was determined by measuring the total fluorescence intensity. For binding analysis, the lysate was diluted fourfold with ECF (containing 0.1% Tween-20) to achieve optimal conditions of fluorescence protein in the binding detection. Starting from the highest concentration of 1 mM, the compound was serially diluted 16 times in ECF at a 1:1 ratio. Subsequently, 5 μl of cell lysate was mixed with 5 μl of different concentrations of the compound. After incubating at room temperature for 5 minutes, all samples were loaded into MST NT.115 standard glass capillaries and measured at 70-100% excitation power. The thermophoresis time was set to 23 s, and the experimental temperature was maintained at 25° C. The experiment was repeated in at least three independent trials, and the data was imported into the MO.NanoTemper affinity analysis software, using the K_{d} model to calculate the value of the K_{d}. Cold fluorescence and hot fluorescence were measured at −1 to 0 seconds and 4 to 5 seconds, respectively, to avoid heat-induced protein conformational changes.

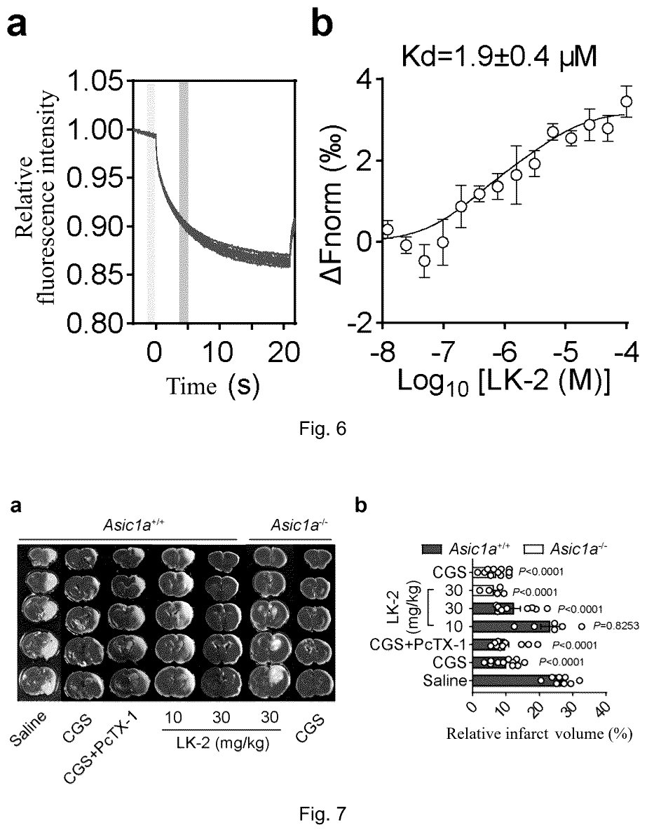

The results of microscale thermophoresis detection (see FIG. 6) show that the K_{d} value of LK-2 binding to ASIC1a was 1.9÷0.4 μM (at pH 7.0), indicating that LK-2 can significantly bind to ASIC1a protein with affinity at the micromolar level.
3.3 Pharmacokinetic Study Solution of LK-2: analysis of the pharmacokinetics of LK-2 (single intraperitoneal injection, 30 mg/kg) in male C57BL/6J mice (n=22). LC-MS/MS method was used to measure the concentration of LK-2 in plasma and brain tissue. LK-2 was dissolved in 0.9% saline to a concentration of 1 mg/ml. Blood samples were collected at 5 min, 15 min, 30 min, 1.0 h, 2.0 h, and 4.0 h time points into EDTA-coated tubes, then centrifuged at 2,000 g for 15 min to generate plasma samples. Brain tissue samples were collected after perfusion with saline through the left ventricle, and the brain tissue were diluted and homogenized with 5 volumes (w:v) of 0.9% NaCl. LC-MS/MS method for quantification of LK-2 in plasma and brain tissue samples was developed using LC-MS/MS-T_API4000 instrument. The general sample processing procedure is as follows.
1) 30 μl plasma samples were divided, standard samples were calibrated with quality control, and single blank and double blank samples were added into 1.5 ml tubes, respectively. 40 μl brain tissue homogenate were added to each well of a 96-well plate, respectively, and standard samples were calibrated with quality control, single blank and double blank samples were aliquoted.
2) Each sample (excluding double blanks) was mixed with 150 μl (plasma samples) or 200 μl (brain tissue homogenate) of internal standard solution [a 6-in-1 internal standard solution (100 ng/ml each of labetalol, tolbutamide, verapamil, dexamethasone, glipizide, and celecoxib dissolved in methanol) and 40 mM dibutyl acetic acid] to terminate the reaction (double blank samples were terminated with 40 mM dibutyl acetic acid in methanol). The mixture was then thoroughly vortexed for at least 15 s and centrifuged at 12000 g (plasma samples) or 3220 g (brain homogenate) at 4° C. for 15 minutes.
3) 65 μl of the supernatant was evenly transferred into a 96-well plate and centrifuge at 3220 g, 4° C. for 5 minutes. All procedures were performed on wet ice. All the supernatant was directly injected for LC-MS/MS analysis. The chromatographic column was ACQUITY UPLC BEHC18 with 2.1×100 mm, 1.7 μm. The column temperature was at 40° C. and the flow rate was 0.4 mL/min. The mobile phase A was 0.001% NH₃·H₂O, 0.18 mM dibutylamine acetate aqueous solution, and mobile phase B was 10 mM N,O-dimethylhydroxylamine and 3 mM NH₄OAc solution, acetonitrile/aqueous solution (v:v, 50:50). Standard curves were prepared by spiking compounds into control plasma and brain to determine drug concentrations. Pharmacokinetic parameters and average concentrations at each time point were calculated using DAS 2.0 software through non-compartmental analysis (NCA), as shown in Table 1.

TABLE 1
Pharmacokinetic parameters of single
intraperitoneal injection of LK-2

| | i.p. 30 mg/kg | |
|---|---|---|
| Parameter | Plasma | Brain tissue |
| AUC₍₀₋ₜ₎ (μg/L*h) | 69793.793 ± 3116.495 | 523.378 ± 27.885 |
| AUC_{(0-∞)} (μg/L*h) | 70278.227 ± 3131.89 | 792.881 ± 68.039 |
| MRT₍₀₋ₜ₎ (h) | 0.693 ± 0.019 | 1.762 ± 0.078 |
| MRT_{(0-∞)} (h) | 0.721 ± 0.018 | 3.63 ± 0.611 |
| t_{1/2} (h) | 0.576 ± 0.015 | 2.297 ± 0.441 |
| tₘₐₓ (h) | 0.5 | 0.5 |
| CL (L/h/kg) | 0.428 ± 0.019 | 38.042 ± 3.197 |
| V (L/kg) | 0.356 ± 0.021 | 124.755 ± 15.127 |
| Cₘₐₓ (μg/L) | 92660 ± 8312.196 | 235.875 ± 31.904 |

| | |
|---|---|
| Brain/plasma AUC ratio | 0.0127 ± 0.0006 |

Note:
AUC: Area under the curve; MRT: Mean residual time; tₘₐₓ: Time to reach maximum plasma concentration; CI: Clearance rate; V: Volume of distribution; Cₘₐₓ: Maximum plasma concentration.

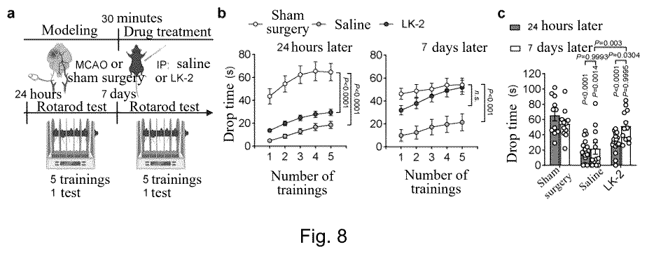

3.4 The animal model of cerebral ischemic stroke, known as the middle cerebral artery occlusion (MCAO) model, is a classic model used to simulate individual patients with ischemic stroke, and evaluate the size of cerebral infarction and motor function (evaluate through the rotarod test, including assessment of motor learning and coordination, the experimental design process is illustrated in FIG. 8a) in model animals after drug intervention. The specific steps are as follows: a transient focal ischemic model was formed by occluding the middle cerebral artery for 30 minutes in wild-type or ASIC1a gene knockout mice. The animals were anesthetized using the animal anesthesia machine (RWD). During the induction of anesthesia, the animals are placed in a transparent chamber (15×25×30 cm) containing 2% isoflurane. During the MCAO procedure, the animals are worn a mask delivering 1% isoflurane. Complete occlusion of the middle cerebral artery was confirmed using continuous laser Doppler blood flow measurement (Moor, FLPI-2). Animals with less than 50% reduction in blood flow during MCAO were excluded. The temperature of rectal and temporal muscle was maintained at 33±0.5° C., using a temperature-controlled heating pad to maintain animal body temperature. Intraperitoneal injection of drugs was performed immediately after removal of the suture occlusion. Animals were euthanized with excess isoflurane 24 hours after ischemia, and brain tissue was rapidly removed and coronally sliced at 1 mm intervals and stained with the vital dye 2,3,5-triphenyltetrazolium chloride (TTC), with normal brain tissue staining red with TTC. The calculation method for infarct volume (%) was to add up the infarct areas of all sections and multiply by the slice thickness, then divide by the entire brain volume. The procedures and analysis were conducted in a blinded manner. The motor function test involved using a rotarod apparatus (RWD) to assess motor coordination and motor learning behavior. In this test, mice were placed on the rotating rod and the time to fall was recorded. Mice were trained 5 times to learn to move on the rod, and then tested with an acceleration from 4 to 40 rpm within 3 minutes. The rotarod rod was cleaned with alcohol before each test.

In the MCAO model animals, mice were intraperitoneally injected with LK-2 at a dose of 30 mg/kg, the volume of cerebral infarction was reduced by 52.8% (see FIG. 7). Seven days after brain injury, mice in the LK-2 group showed significant improvement in motor learning ability, with an overall improvement of 51.6% in motor function (FIGS. 8b and c).

### Example 2

This example provides a synthetic method for the small molecule compound LK-2, which includes the following steps:

Step 1: Synthesis of 1-benzyl-2-methyl-4-((dimethoxyphosphinyl)(phenyl)methyl)piperazine-1,2-dicarboxylate: 2-methylpiperazine-1,2-dicarboxylic acid benzyl ester (330 mg, 1.19 mmol) was dissolved in acetonitrile (15 mL), benzaldehyde (138 mg, 1.3 mmol), and dimethyl phosphite (193.3 mg, 1.54 mmol). The reaction mixture was heated at 80° C. for 48 hours. The reaction was monitored by TLC and the solvent was removed under reduced pressure using a rotary evaporator when the reaction ended. The resulting residue was purified by column chromatography (SiO₂, DCM/MeOH=20:1) to obtain 1-benzyl-2-methyl-4-((dimethoxyphosphinyl)(phenyl)methyl)piperazine-1,2-dicarboxylate. Yield: 400 mg (70%). m/z: ES⁺[M+H]⁺=477.1.

Step 2: Synthesis of 4-(phenyl(phosphinoyl)methyl)piperazine-2-carboxylic acid (LK-2): 1-benzyl-2-methyl-4-((dimethoxyphosphoryl)(phenyl)methyl)piperazine-1,2-dicarboxylate (400 mg, 0.840 mmol) was dissolved in concentrated hydrochloric acid (6 N, 20 ml), and the reaction mixture was then heated to 90° C. for 24 hours. When the reaction reaches the end under the monitoring of TLC, the reaction mixture was cooled to room temperature and concentrated to dryness under reduced pressure using a rotary evaporator. The resulting residue was purified by reverse-phase chromatography to obtain 4-(phenyl(phosphinoyl)methyl)piperazine-2-carboxylic acid, which was a white solid, under the following conditions: chromatographic column, C18, 40 g, 20-35 μm, 100 Å; mobile phase, CH₃CN: H₂O (0.05% NH₃·H₂O)=5:95; detector, UV 200 nm. Yield: 170 mg (68%). m/z: ES⁺[M+H]⁺=301.1.

¹H NMR (400 MHz, D₂O) δ 7.52-7.36 (m, 5H), 4.48-4.40 (m, 1H), 4.30-4.08 (m, 2H), 3.79-3.54 (m, 2H), 3.46-3.22 (m, H).

The above has described embodiments of the present invention in detail, but they are only examples and the present invention is not limited to the embodiments described above. For those skilled in the art, any equivalent modifications and substitutions to the present invention are also within the scope of the present invention. Therefore, all equivalent transformations and modifications made without departing from the spirit and scope of the present invention should be encompassed within the scope of the present invention.

What is claimed is:

1. A small molecule compound for treating ischemic stroke, wherein the small molecule compound is 4-(phenyl (phosphinoyl) methyl) piperazine-2-carboxylic acid, and its structural formula is as follows:

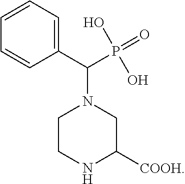 [SMILES: O=C(O)C1CN(C(c2ccccc2)P(=O)(O)O)CCN1]

2. A method for preparing the small molecule compound as claimed in claim 1, comprising:
step 1, synthesis of 1-benzyl-2-methyl-4-((dimethoxyphosphoryl)(phenyl)methyl)piperazine-1,2-dicarboxylate: dissolving 2-methylpiperazine-1,2-dicarboxylate benzyl ester in an organic solvent, benzaldehyde, and dimethyl phosphite, and reacting to obtain 1-benzyl-2-methyl-4-((dimethoxyphosphoryl)(phenyl)methyl)piperazine-1,2-dicarboxylate;
step 2, synthesis of 4-(phenyl(phosphinoyl)methyl)piperazine-2-carboxylic acid: dissolving 1-benzyl-2-methyl-4-((dimethoxyphosphoryl)(phenyl)methyl)piperazine-1,2-dicarboxylate in concentrated hydrochloric acid, and reacting to obtain 4-(phenyl(phosphinoyl)methyl)piperazine-2-carboxylic acid.

3. The method of claim 2, wherein the organic solvent is acetonitrile.

4. The method of claim 2, wherein in step 1, the molar ratio of 2-methylpiperazine-1,2-dicarboxylate benzyl ester, benzaldehyde, and dimethyl phosphite is 1:1˜1.5:1.25˜1.80; preferably 1:1.09:1.29.

5. The method of claim 2, wherein the reaction condition for step 1 is at 70˜90° C. lasting for 36˜60 hours, preferably at 80° C. lasting for 48 hours.

6. The method of claim 2, wherein in step 2, the molar ratio of 1-benzyl-2-methyl-4-((dimethoxyphosphoryl)(phenyl)methyl)piperazine-1,2-dicarboxylate to hydrochloric acid is 5˜10:1, preferably 7:1.

7. The method of claim 2, wherein the reaction condition for step 2 is at 80˜100° C. lasting for 18˜36 hours, preferably at 90° C. lasting for 24 hours.

8. The method of claim 2, wherein step 1 further comprises: when the reaction reaches its end, the solvent is removed by using a rotary evaporator under reduced pressure; the resulting residue is purified by column chromatography.

9. The method of claim 2, wherein step 2 further comprises: when the reaction reaches its end, the reaction mixture is cooled to room temperature, concentrated to dryness by using a rotary evaporator under reduced pressure; the resulting residue is purified by reverse-phase chromatography.

10. The application of the small molecule compound as claimed in claim 1 in the preparation of a pharmaceutical for treating ischemic stroke, wherein the pharmaceutical comprises the small molecule compound which functions as an effective active ingredient.

11. A competitive antagonist of acid-sensing ion channel proteins (ASICs), comprising the small molecule compound as claimed in claim 1.

12. A pharmaceutical or pharmaceutical composition for treating ischemic stroke, comprising the small molecule compound as claimed in claim 1 which functions as an effective active ingredient.
